SEQUENCE LISTING (24 sequences)

SEQ ID NO: 1 - RNA, 41 nt, synthetic construct
  source 1..41: mol_type = other RNA
taatttctactaagtgtagatgttttgtcctcaggccccta

SEQ ID NO: 2 - RNA, 41 nt, synthetic construct
  source 1..41: mol_type = other RNA
taatttctactaagtgtagatgcttccactccccttcctct

SEQ ID NO: 3 - RNA, 41 nt, synthetic construct
  source 1..41: mol_type = other RNA
taatttctactaagtgtagatttaatgaattaatgaaagtg

SEQ ID NO: 4 - RNA, 41 nt, synthetic construct
  source 1..41: mol_type = other RNA
taatttctactaagtgtagatgtttcgtcctcaggccccta

SEQ ID NO: 5 - RNA, 41 nt, synthetic construct
  source 1..41: mol_type = other RNA
taatttctactaagtgtagatggctccattccccttcctct

SEQ ID NO: 6 - RNA, 41 nt, synthetic construct
  source 1..41: mol_type = other RNA
taatttctactaagtgtagatttaacgaattaatgaaagtg

SEQ ID NO: 7 - DNA, 24 bp, synthetic construct
  source 1..24: mol_type = other DNA
cgtgtattacgaaggagaacacaa

SEQ ID NO: 8 - DNA, 22 bp, synthetic construct
  source 1..22: mol_type = other DNA
aagatgcttggccctcccacag

SEQ ID NO: 9 - DNA, 26 bp, synthetic construct
  source 1..26: mol_type = other DNA
gggttttgggctccaagagatttggg

SEQ ID NO: 10 - DNA, 21 bp, synthetic construct
  source 1..21: mol_type = other DNA
gtgaggttgaatggcctgtcc

SEQ ID NO: 11 - DNA, 20 bp, synthetic construct
  source 1..20: mol_type = other DNA
tgaatgtggcctatgctggt

SEQ ID NO: 12 - DNA, 20 bp, synthetic construct
  source 1..20: mol_type = other DNA
acagtggctggtcatcatac

SEQ ID NO: 13 - RNA, 41 nt, synthetic construct
  source 1..41: mol_type = other RNA
taatttctactaagtgtagatgtttcgtccccaggccccta

SEQ ID NO: 14 - RNA, 41 nt, synthetic construct
  source 1..41: mol_type = other RNA
taatttctactaagtgtagatgttttgtccccaggccccta

SEQ ID NO: 15 - RNA, 41 nt, synthetic construct
  source 1..41: mol_type = other RNA
taatttctactaagtgtagatggctccactccccttcctct

SEQ ID NO: 16 - RNA, 41 nt, synthetic construct
  source 1..41: mol_type = other RNA
taatttctactaagtgtagatggttccactccccttcctct

SEQ ID NO: 17 - RNA, 41 nt, synthetic construct
  source 1..41: mol_type = other RNA
taatttctactaagtgtagatacgaattaatgaaagtggat

SEQ ID NO: 18 - RNA, 41 nt, synthetic construct
  source 1..41: mol_type = other RNA
taatttctactaagtgtagatatgaattaatgaaagtggat

SEQ ID NO: 19 - DNA, 30 bp, synthetic construct
  source 1..30: mol_type = other DNA
acataaaatacaagattttgcagaattttg

SEQ ID NO: 20 - DNA, 19 bp, synthetic construct
  source 1..19: mol_type = other DNA
gtttccctcagccactgcc

SEQ ID NO: 21 - DNA, 26 bp, synthetic construct
  source 1..26: mol_type = other DNA
gggttttgggctccaagagatttggg

SEQ ID NO: 22 - DNA, 24 bp, synthetic construct
  source 1..24: mol_type = other DNA
ccagcctgggtaacagagcaagac

SEQ ID NO: 23 - DNA, 35 bp, synthetic construct
  source 1..35: mol_type = other DNA
gaagtagttttctcatttttaaatgacaaaattta

SEQ ID NO: 24 - DNA, 19 bp, synthetic construct
  source 1..19: mol_type = other DNA
gccagcctgggcaacagag

(19) United States
(12) Patent Application Publication
(10) Pub. No.: US 2024/0401111 A1
(43) Pub. Date: Dec. 5, 2024

(54) ASSAY DEVICE, METHOD FOR DETERMINING GENETIC PREDISPOSITION TO SEVERE FORMS OF SARS-COV2 INFECTION

(71) Applicant: CRISPRBITS PRIVATE LIMITED, New Delhi (IN)

(72) Inventors: Vijay CHANDRU, Bangalore (IN); Sunil ARORA, Delhi (IN); Bharat JOBANPUTRA, Edmonton (CA); Aditya SARDA, Panchkula (IN); Suruchi SHARMA, Bangalore (IN); Vaijayanti GUPTA, Bangalore (IN); Nimisha GUPTA, Bangalore (IN); Satish SANKARAN, Bangalore (IN); Annes SIJI, Bangalore (IN); Manasa Bagur PRAKASH, Bangalore (IN)

(73) Assignee: CRISPRBITS PRIVATE LIMITED, New Delhi, DL (IN)

(21) Appl. No.: 18/697,463

(22) PCT Filed: Sep. 30, 2022
(86) PCT No.: PCT/IB2022/059355

§ 371 Date: Mar. 30, 2024

(30) Foreign Application Priority Data
Sep. 30, 2021 (IN) 202111014843

(51) Int. Cl. C12Q 1/6818; C12Q 1/6858; C12Q 1/6876

(52) U.S. Cl. CPC C12Q 1/6818; C12Q 1/6858; C12Q 1/6876; C12Q 2600/118; C12Q 2600/156

(57) ABSTRACT

The present disclosure provides an assay device and associated method(s) to facilitate the determination of risk of a subject to develop severe forms of SARS-CoV2 infection. Said determination of risk or genetic predisposition to severe forms of SARS-CoV2 infection employing the assay device and associated method(s) relies on specifically designed CRISPR-Cas system(s). Said CRISPR-Cas system(s) employed in the present disclosure are further characterized by unique guide sequences that enable detection of presence or absence of specific Single Nucleotide Polymorphisms (SNPs) in a sample, wherein the SNPs have a genetic correlation with severe forms of SARS-CoV2 infection.

### TECHNICAL FIELD

The present disclosure relates to the fields of molecular biology, genetics, disease management and patient management. Particularly, the present disclosure provides an assay device and associated method(s) to facilitate the determination of risk of a subject developing severe forms of SARS-CoV2 infection. In some embodiments, said determination employing the assay device and associated method(s) rely on specifically designed CRISPR-Cas system(s), characterized by unique guide sequences that enable detection of presence or absence of specific Single Nucleotide Polymorphisms (SNPs) in a sample, wherein said SNPs have a genetic correlation with severe forms of SARS-CoV2 infection.

### BACKGROUND OF THE DISCLOSURE

The wide range of severity seen in the population infected with respiratory infection such as COVID-19 underscores the importance of host factors in progression and manifestation of the disease. Various efforts have been directed to identify the genetic factors that can be linked to the severity of such infections.

The knowledge of host genetic factors that are associated with the severity of the infections can help in managing the infections in several ways. Said knowledge can be used as basis to identify new drug targets, and hence enable drug repurposing. If genetic factors can be used to ascertain the mechanism or pathway involved in the disease progression, the existing drugs known to work in that pathway could be repurposed for the treatment of severe forms of certain respiratory infections. The genetic association of response to a given therapy and extent of predisposition to the severity of the disease, can help improve the preparedness for the individual and significantly improve the patient management. This is particularly helpful in deciding on and prioritizing the course of treatment in pediatric cases.

Additionally, the knowledge of genetic predisposition can be used for policy making; for example, in deciding the priority groups for vaccination, more so in case of new infections such as COVID-19, wherein the vaccine is in the preliminary stages of being rolled out for the population at large.

Further, vaccines that are being newly developed are not free from adverse reactions in certain individuals. There is a reasonable probability that host genetic factors may be associated with the adverse reactions to the vaccine. Identifying those genetic factors would therefore significantly address side effects of vaccines and help avoid the same in susceptible individuals.

Accordingly, in order to reap the benefits of identification of host genetic factors that determine severity of respiratory infections such as SARS-CoV2 infection, there exists a need for accessible and affordable tests that allow for large scale testing of these genetic factors.

### SUMMARY OF THE DISCLOSURE

Addressing the aforesaid need in the art, the present disclosure provides an assay device for determining genetic predisposition to severe forms of SARS-CoV2 infection comprising at least one CRISPR-Cas system for genotyping of at least one Single Nucleotide Polymorphism (SNP) selected from a group comprising rs10735079, rs2109069 and rs73064425 or any combination thereof in a biological sample.

In some embodiments, the device comprises separate CRISPR-Cas systems for detection of each SNP. In some embodiments, CRISPR-Cas system has sgRNA sequence selected from a group comprising sequences represented by SEQ ID Nos. 1-6.

In some embodiments, the CRISPR-Cas system has sgRNA sequence selected from a group comprising sequences represented by SEQ ID Nos. 1-3 for confirming presence of risk allele(s) for one or more SNPs selected from a group comprising rs10735079, rs2109069 and rs73064425 or any combination thereof.

In some embodiments, the CRISPR-Cas system has sgRNA sequence selected from a group comprising sequences represented by SEQ ID Nos. 4-6 for confirming presence of alternate allele(s) for one or more SNPs selected from a group comprising rs10735079, rs2109069 and rs73064425 or any combination thereof.

In some embodiments, the CRISPR-Cas system comprises Cas nuclease selected from a group comprising dCas9, Cas9, Cas-Phi, Cas12, Cas12a, Cas12b and Cas13a or any combination thereof. In an exemplary embodiment, the Cas nuclease is Cas12a; and wherein the detection is facilitated by trans-cleavage mechanism of the Cas12a nuclease.

In some embodiments, the assay device further comprises reporter molecule(s) in the format of F—(N)ₙ-Q or Q-(N)ₙ—F; wherein F is a fluorescent reporter molecule; wherein N is selected from A, G, T, C, rA, rG, rT and rC; and wherein Q is a quencher.

In some embodiments, the fluorescent reporter molecule is selected from a group comprising SYT09, fluorescein, rhodamine, rhodoamine600, R-phycoerythrin, and Texas Red or any combination thereof; wherein the quencher is selected from a group comprising black hole quencher (BHQ), Iowa black and 4-(dimethylaminoazo)benzene-4-carboxylic acid (DABCYL); and wherein n ranges from about 6 to about 15.

In some embodiments, the at least one CRISPR-Cas system is immobilized on the surface of the assay device.

Further provided herein is a method of detecting genetic predisposition to severe forms of SARS-CoV2 comprising adding a biological sample to the assay device as described above to obtain a detectable signal.

In some embodiments, the sample comprises nucleic acid; and wherein the nucleic acid is subjected to amplification before its addition to the assay device.

In some embodiments, the amplification is performed by Polymerase Chain Reaction (PCR); wherein primer(s) for the PCR amplification is selected from a group comprising sequences represented by SEQ ID Nos. 7-12.

In some embodiments, in the assay device or the method as described above, the genetic predisposition to severe forms of SARS-CoV2 infection is determined by presence of risk and/or alternate allele(s) for SNPs selected from a group comprising rs10735079, rs2109069 and rs73064425 or any combination thereof in the biological sample.

Further provided in the present disclosure is sgRNA sequence against loci of SNP(s) selected from a group comprising rs10735079, rs2109069 and rs73064425 or any combination thereof.

In some embodiments, the sgRNA has sequence selected from a group comprising sequences represented by SEQ ID Nos. 1-6.

Further envisaged herein is the sgRNA sequence as described above, for use in detecting genetic predisposition to severe forms of SARS-CoV2.

Further provided in the present disclosure is use of a device comprising at least one CRISPR-Cas system for detection of at least one Single Nucleotide Polymorphism (SNP) selected from a group comprising rs10735079, rs2109069 and rs73064425 or any combination thereof in a biological sample or the sgRNAs as defined above or a CRISPR-Cas complex comprising the sgRNAs as defined above in conjunction with Cas12a nuclease in detecting genetic predisposition to severe forms of SARS-CoV2.

The present disclosure further provides a kit comprising the assay device or the sgRNA sequences as defined above, along with an instruction manual.

In some embodiments, the kit further comprising one or more component(s) selected from a group comprising a multi-well plate, reaction buffer(s), means for sample procurement, means for application of the sample to the assay device, primer(s), dNTPs, polymerase enzyme(s) and reporter system(s) or any combination thereof.

### BRIEF DESCRIPTION OF THE ACCOMPANYING FIGURES

In order that the disclosure may be readily understood and put into practical effect, reference will now be made to exemplary embodiments as illustrated with reference to the accompanying figures. The figures together with detailed description below, are incorporated in and form part of the specification, and serve to further illustrate the embodiments and explain various principles and advantages, in accordance with the present disclosure where:

FIG. 1. provides a schematic diagram for the genotype determination assay.

FIG. 2. provides results of the trans-cleavage assay on the synthetic targets for detection of the three SNPs rs10735079, rs2109069 and rs73064425 employing the guides selected for the assay and provided in Table C.

FIG. 3. provides results of the trans-cleavage assay on the synthetic targets for detection of the three SNPs rs10735079, rs2109069 and rs73064425 employing the comparative/alternate guides, representative of other tested primers, as provided in Table D.

FIG. 4. provides gel electrophoresis results for (a) amplification of the SNPs employing primers as provided in Table C; (b) multiplexed amplification of the SNPs using the primers as provided in Table C and (b) amplification of the SNPs using the primers as provided in Table D.

FIG. 5. (i) provides results of the trans-cleavage assays performed to ascertain the genotype of NA12878 using the guide pair designed for each SNP after individual amplification of the genomic region harboring each SNP; (ii) provides results of the trans-cleavage assays performed to ascertain the genotype of NA12878 using the guide pair designed for each SNP after multiplexed amplification of the genomic regions harboring the indicated SNP in a single tube.

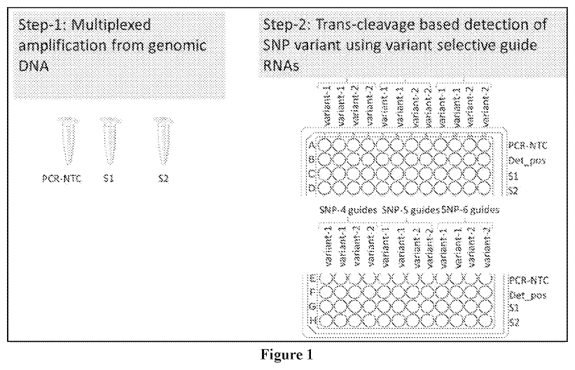

### DETAILED DESCRIPTION OF THE INVENTION

Addressing the aforesaid need pertaining to utilizing the potential in identification of host genetic factors that determine severity of respiratory infections, the present disclosure provides means for efficient and speedy detection of specific SNPs in individuals to enable determination of genetic predisposition to severe forms of respiratory infections.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. A number of terms are defined and used throughout the specification with the following definitions provided for convenience.

### Definitions

“Single Nucleotide Polymorphism” or “SNP” refers to a variation of a single base in the genome of a living organism. SNPs may occur in coding sequences of genes and non-coding regions (including regulatory regions) of genes. SNPs in genomic coding sequences are classified into two types: synonymous and non-synonymous. The SNP nomenclature as reported herein refers to the official Reference SNP (rs) ID identification tag as assigned to each unique SNP by the National Center for Biotechnological Information (NCBI).

As used herein the terms “assay device” or “device” as used interchangeably refer to the CRISPR-Cas based assay platform of the present disclosure to which the sample is applied and that provides a detectable signal or readout allowing for detection of presence or absence of specific SNPs in the sample.

A person skilled in the art will appreciate that for markers with two alleles present in the population being studied (such as SNPs), and wherein one allele is found in increased frequency in a group of individuals with a trait or disease in the population, compared with controls, the alternative allele of the marker will be found in decreased frequency in the group of individuals with the trait or disease, compared with controls. In such a case, one allele of the marker (the one found in increased frequency in individuals with the trait or disease) will be the “at-risk” or “risk allele”, while the other allele will be the “alternative allele”. Determination of the absence of any one of these risk alleles is indicative that the individual does not have the increased risk conferred by the allele.

In the context of the present disclosure, “risk allele” is indicative of the presence of the SNP of interest—which determines genetic predisposition to severe forms of SARS-CoV2. Further, “alternative allele” as referred to herein refers to absence of the SNP of interest and therefore essentially implies an allele that is not a risk allele.

Most SNP polymorphisms have two alleles. Each individual accordingly is either “homozygous” for one allele of the polymorphism (i.e. both chromosomal copies of the individual have the same nucleotide at the SNP location), or the individual is “heterozygous” (i.e. the two sister chromosomes of the individual contain different nucleotides) The terms “homozygous” and/or “heterozygous” have therefore been used in the said context in the present disclosure.

As used herein, a “primer” (primer sequence) is a short oligonucleotide of an appropriate length sufficient to hybridize to a target DNA (e.g., a single-stranded DNA) and allow addition of nucleotide residues thereto, or an oligonucleotide or polynucleotide synthesized therefrom under suitable conditions well known in the art. The primer is designed to have a sequence of complement that is the region of the template/target DNA to which the primer hybridizes.

As used herein, the term “subject” denotes a mammal. Preferably reference to a “subject” in the present disclosure implies a human subject.

As used herein, a “sample” refers to biological sample from a subject which may comprise the target for which the analysis method according to the embodiment should be performed. The sample is preferably in a state that does not impede amplification reaction and/or hybridization reaction. For example, to use a material obtained from a living body as a sample according to the embodiment, the material may have to pre-processed using a certain means. The sample may be a diseased or a non-diseased sample.

A nucleic acid contained in a sample is called a “sample nucleic acid” or simply “nucleic acid” in the context of the present disclosure. The term “nucleic acid” assumes ordinary meaning of the said term that is conventionally used and well known to a person skilled in the art. It refers to any one or more nucleic acid segments. Out of the sample nucleic acid, a sequence to be amplified by a primer according to the embodiment is called a “template sequence” or a “template”.

“Amplified nucleic acid” refers to an amplified product obtained by amplifying the SNP locus from nucleic acid isolated from a sample. “Target loci” refers to the loci of the SNPs that are to be detected by the assay device of the present disclosure.

“Amplification mix” in the context of the present disclosure refers to the mix of template, dNTPs, primer(s), buffer(s), polymerase enzyme(s) and the amplified DNA (preferably amplified at target loci) obtained at the end of the amplification cycle that the nucleic acids are subjected to, before application to the assay device of the present disclosure.

The term “CRISPR” as used throughout the present disclosure is an abbreviation for Clustered Regularly Interspaced Short Palindromic Repeats and refers to the conventional CRISPR technology well known to a person skilled in the art. CRISPR (clustered regularly interspaced short palindromic repeats) are short stretches of DNA sequences discovered in the genomes of prokaryotes, derived from invading phages that attacked them previously. They serve as a memory to destroy similar phages during subsequent infections. More recently, exploiting the high precision and accuracy of these genetic scissors, the technology has been optimized to edit genes in vitro or in vivo, for research as well as therapeutic applications, for treating diseases with clear genetic basis.

The term “CRISPR-Cas system” and obvious variants thereof as referred to in the present disclosure refers to the collection of elements comprising but not limited to a Cas protein and a guide-polynucleotide.

The terms “CRISPR guide”, “guide sequence”, “guide RNA” and obvious variants thereof as referred to in the present disclosure refers to the guide-polynucleotide of the CRISPR-Cas system, wherein in the present disclosure, the CRISPR guide is designed to recognize the presence of absence of the SNP(s) of interest at the target loci. In some embodiments, the CRISPR guide is designed as a dual crRNA:tracrRNA guide or a single-molecule guide RNA.

The term “single guide RNA” or “sgRNA” as used in the present disclosure is in reference to a single RNA molecule that is sufficient to bind to LbaCas12a and the corresponding DNA target to initiate the trans-cleavage of a ssDNA reporter.

As used herein, the term “reporter” or “reporter system” refers to a “label” that can be used to provide a detectable (preferably quantifiable) signal. Reporters may provide signals detectable by fluorescence, luminescence, radioactivity, colorimetry, X-ray diffraction or absorption, magnetism, enzymatic activity, and the like. “Activation” of the reporter system refers to the modification of the reporter system in a way that yields a “detectable signal” or “readout”, indicating the presence or absence of the SNP of interest.

The term “detection event” as used herein refers to subjecting the sample to recognition by a specific guide RNA, wherein each guide RNA is designed to recognize the presence of a risk allele or an alternative allele in a sample. Accordingly, guide RNA is designed to facilitate a separate detection event, for determining the presence of a risk allele or an alternative allele. In some embodiments, the design of the assay device of the present disclosure is such that every detection event yields a detectable signal, confirming presence of the risk allele (interchangeably referred to herein as a ‘positive detection event’) or the alternative allele (interchangeably referred to herein as a ‘negative detection event’) in the tested sample. Thus, one or more guides are designed to confirm the presence or absence of an SNP, such that one guide is directed against the risk allele containing the SNP and additional guide(s) are directed against the alternative allele(s) that do not carry the SNP. Said separate detection event in non-limiting embodiments, may occur in separate tubes, wells on the device surface, lateral flow strips, uniquely tagged beads or in multiplexed reactions.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The use of the expression ‘at least’ or ‘at least one’ suggests the use of one or more elements or ingredients or quantities, as the use may be in the embodiment of the disclosure to achieve one or more of the desired objects or results. Numerical ranges stated in the form ‘from x to y’ include the values mentioned and those values that lie within the range of the respective measurement accuracy as known to the skilled person. If several preferred numerical ranges are stated in this form, of course, all the ranges formed by a combination of the different end points are also included.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically. and preferably, disclosed.

As used herein, the terms “include” (any form of “include”, such as “include”), “have” (and “have”), “comprise” etc. any form of “having”, “including” (and any form of “including” such as “including”), “containing”, “comprising” or “comprises” are inclusive and will be understood to imply the inclusion of a stated element, integer or step, or group of elements, integers or steps, but not the exclusion of any other element, integer or step, or group of elements, integers or steps

It is to be noted that the term “a” or “an” entity refers to one or more of that entity. As such, the terms “a” (or “an”), “one or more”, and “at least one” can be used interchangeably herein.

As regards the embodiments characterized in this specification, it is intended that each embodiment be read independently as well as in combination with another embodiment. For example, in case of an embodiment 1 reciting 3 alternatives A, B and C, an embodiment 2 reciting 3 alternatives D, E and F and an embodiment 3 reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

### Disclosure

The present disclosure is based on the physiopathological impact of specific Single Nucleotide Polymorphisms (SNPs) on the severity of SARS-CoV2 infection. The present disclosure is centered around the detection of said SNPs in a sample to enable efficient patient management and disease management by predicting genetic predisposition of individuals to severe forms of SARS-CoV2 infection. The present disclosure provides means for said detection.

Specifically, in order to facilitate said detection, the present disclosure provides an assay device that allows CRISPR mediated detection of SNPs that predispose an individual to severe forms of SARS-CoV2 infection, characterized by incorporation of specifically designed CRISPR guide sequences. Said unique guide sequences paired with Cas nucleases are employed as part of a tailored assay designed to detect the presence or absence of SNPs in a sample to determine the extent of genetic predisposition of a subject to severe forms of specific respiratory infections.

Accordingly, provided herein is an assay device comprising a CRISPR-Cas system for detection of SNP(s) of interest in a sample. Particularly, the present disclosure provides an assay device that allows CRISPR mediated detection genotyping of SNPs that indicate genetic predisposition of a subject to severe forms of SARS-CoV2 infection.

More specifically, the present disclosure provides an assay device that allows CRISPR mediated genotyping of SNPs that indicate genetic predisposition of a subject to severe forms of SARS-CoV2 infection.

In some embodiments, the assay device is designed to comprise a patterned surface suitable for immobilization of molecules in an ordered pattern. In some embodiments a patterned surface refers to an arrangement of different regions in or on an exposed layer of a solid support. In some embodiments, the solid support comprises an array of wells or depressions on a surface or a lateral flow strip made of paper or any membrane. The composition and geometry of the solid support can vary with its use. In some embodiments, the solid support is a planar structure such as a slide, chip, flow strip, microchip and/or array.

In some embodiments, the assay device is designed in the form of cartridges or reaction vessels, or series or arrays thereof.

In some embodiments, the assay is facilitated on beads or in solution form. Accordingly, in some embodiments, the assay device is composed of beads and means to hold the same or containers comprising the assay reagents in solution, or series or arrays of said containers or set of beads.

In some embodiments, the assay device is made of any material that allows for the immobilization of a polypeptide, a polynucleotide or a protein-nucleic acid complex. In some embodiments, the assay device is made of conductive or non-conductive material. In some embodiments, the assay device is made of non-conductive material and coated with conductive substances to confer to the platform electrical and/or thermal conductivity

In some embodiments, the assay device is made of made of material such as but not limited to glass, modified functionalized glass, plastics, polysaccharides, nylon, nitrocellulose, ceramics, resins, silica, silicon and modified silicon, carbon, metals, inorganic glasses, plastics, optical fiber bundles, and or any other polymers.

The ‘detection’ in the device of the present disclosure is facilitated by exploiting the cleavage efficiency of the CRISPR-Cas system, wherein the guides are designed such that they specifically recognize sequences comprising the SNP(s) of interest or can distinguish two or more targets that vary by a single nucleotide, as seen in the case of single nucleotide polymorphism (SNP), that the present disclosure sets out to detect.

Accordingly, in some embodiments, CRISPR mediated cleavage is considered as a confirmative event for the presence of the target in a sample. In some embodiments, depending on the specific Cas nuclease employed, the assay may utilize the specific or non-specific cleaving activity of the Cas nuclease to render a detectable signal.

In some embodiments, the CRISPR-Cas system as described above, may be immobilized on to the solid substrate or is suspended in liquid medium of the assay device.

In some embodiments, the CRISPR-Cas system directed towards different targets may be immobilized in separate wells or depressions on the surface of the assay device or contained on different sets of beads or in different solutions.

In some embodiments, the CRISPR-Cas system may be present in the assay device in a suspended form in different tube(s) or reaction vessel(s) for each detection event.

In some embodiments, the CRISPR-Cas system may be incorporated into the device in any of the above forms as free nucleotides (guide RNA) and peptides (Cas). In some embodiments, the CRISPR-Cas system may be present in the device in encapsulated form, wherein said encapsulation maybe in vehicles such as but not limited to liposomes or hydrogels.

CRISPR guides employed in the assay device of the present disclosure are designed to recognize and hence facilitate cleavage in the risk allele (confirming presence of the SNP) and/or in the alternative allele (confirming absence of the SNP). Thus, one or more guides are designed to confirm the presence of absence of an SNP, such that one guide is directed against the risk allele containing the SNP and additional guide(s) are directed against the alternative allele(s) that do not carry the SNP.

In some embodiments, the SNP(s) of interest, detection of which is facilitated by the assay device of the present disclosure, includes one or more selected from the below table—

TABLE A

| S. | | Risk variant (predisposition to severe | Alternative |
|---|---|---|---|
| No. | SNP | forms of SARS-CoV2 infection) | variant |
| 1. | rs10735079 | A | G |
| 2. | rs2109069 | A | G |
| 3. | rs73064425 | T | C |

In order to facilitate efficient detection of the SNPs as set out in Table A, the assay device of the present disclosure employs CRISPR guide(s) (sgRNAs) for recognition of presence of a risk allele or an alternate allele for the specific SNPs in the sample, having length between about 38 to about 44 pairs, preferably about 17 to about 20 base pairs, with preference towards shorter guides in view of higher specificity. However, decreasing the length also lowers the sensitivity of the detection. Accordingly, another strategy that is used to increase the specificity is to introduce an additional mismatch in guide RNA at a new position. Notably, both risk and alternative allele containing samples will most likely have the same base at the position of this additional mismatch, but this additional mismatch should not affect the recognition of the alternative allele containing sample. So, the additional mismatch will be introduced in such a position that is known to not decrease the recognition by Cas-guide RNA complex. This additional mismatch on its own does not decrease the recognition, but in presence of mismatch at the position of SNP it cooperatively lowers the recognition and hence improves the specificity of SNP detection. Accordingly, in some embodiments, specificity of the guide RNA is further increased by introducing additional mismatches between the guide RNA and the target such that additional mismatch does not alter the cleavage of the target that does not have a mismatch at the position of SNP. Thus, in some embodiments, the guide RNA is designed to have a synthetic mismatch that is introduced upstream or downstream of the naturally occurring SNP.

In order to detect the presence of one or more of the above listed SNPs, CRISPR guides employed in the assay device are designed to recognize and hence facilitate cleavage in the risk allele) and/or in the alternative allele.

Since the CRISPR guides are designed so as the recognize the risk allele and/or the alternative allele, in some embodiments, the CRISPR-Cas system employed in the assay device of the present disclosure is designed to provide allele specific recognition so as to recognize the zygosity of alleles and determine if an individual is homozygous or heterozygous for the risk allele.

Taken together, the present disclosure provides an assay device for determining genetic predisposition to severe forms of SARS-CoV2 infection comprising at least one CRISPR-Cas system for genotyping of at least one Single Nucleotide Polymorphism (SNP) selected from a group comprising rs10735079, rs2109069 and rs73064425 or any combination thereof in a biological sample.

In some embodiments, the assay device facilitates genotyping of at least one Single Nucleotide Polymorphism (SNP) selected from a group comprising rs10735079, rs2109069 and rs73064425 or any combination thereof.

In some embodiments, the assay device facilitates detection of risk or alternate alleles for one or more Single Nucleotide Polymorphism(s) (SNPs) selected from a group comprising rs10735079, rs2109069 and rs73064425 or any combination thereof.

In some embodiments, the assay device facilitates detection of risk or alternate alleles for two or more Single Nucleotide Polymorphism(s) (SNPs) selected from a group comprising rs10735079, rs2109069 and rs73064425 or any combination thereof.

In some embodiments, the assay device facilitates detection of risk or alternate alleles for Single Nucleotide Polymorphism(s) (SNPs) rs10735079, rs2109069 and rs73064425.

In some embodiments, the assay device comprises separate CRISPR-Cas system(s) for genotypic of for each SNP.

In some embodiments, for the genotyping for each SNP, the assay device may allow for detection of the risk allele and/or the alternate allele. Each such detection is facilitated by a specific CRISPR-Cas system characterized by a guide sequence targeted towards recognition of the risk allele and/or the alternative allele.

In some events, said detection events are performed in duplicates for validation of results.

In an embodiment, the assay device may optionally comprise means for control detection event(s) for each SNP or common control detection event(s) for all SNPs. In some embodiments, the control detection event(s) comprise a positive and a negative control detection event. In some embodiments, the positive control detection event employs a sample known to contain the risk allele and/or the alternative allele. In some embodiments, the negative control detection event does not employ a sample.

In some embodiments, the assay device is designed such that each detection event occurs in separate wells or depressions on the surface of the assay device, or on separate set of beads or separate solutions within the assay device.

In some embodiments, when the risk allele is a dominant allele, detection of the risk, may alone be relied upon to confirm genetic predisposition to severe form(s) of the SARS-CoV2 infection. In some embodiments, absence the risk allele is confirmed by confirming presence of alternative alleles alone.

In non-limiting embodiments of the present disclosure, in order to facilitate detection of genetic predisposition of severe forms of SARS-CoV2 infection, the present disclosure provides the following CRISPR guide sequences directed against respective SNPs pertinent to SARS-CoV2 infection as indicated above, wherein the sequences provide for detection of the risk allele and the alternative allele.

TABLE B

| | | | Guide |
|---|---|---|---|
| | | | sequence |
| | | | (alternative |
| | | | alleles |
| | | Guide | where |
| | | sequence | alternate |
| | | (risk | nucleotide |
| | | allele- | in |
| | | SNP | bracket |
| S. | | bold | and bold |
| No. | SNP | underlined) | underlined) |
| 1. | rs10735079 | UAAUUUCUAC | UAAUUUCUAC |
| | | UAAGUGUAGA | UAAGUGUAGA |
| | | UGUUUUGUCC | UGUUUCGUCC |
| | | UCAGGCCCCU | UCAGGCCCCU |
| | | A | A |
| | | [SEQ ID | [SEQ ID |
| | | No. 1] | No. 4] |
| 2. | rs2109069 | UAAUUUCUAC | UAAUUUCUAC |
| | | UAAGUGUAGA | UAAGUGUAGA |
| | | UGCUUCCACU | UGGCUCCAUU |
| | | CCCCUUCCUC | CCCCUUCCUC |
| | | U | U |
| | | [SEQ ID | [SEQ ID |
| | | No. 2] | No. 5] |
| 3. | rs73064425 | UAAUUUCUAC | UAAUUUCUAC |
| | | UAAGUGUAGA | UAAGUGUAGA |
| | | UUUAAUGAAU | UUUAACGAAU |
| | | UAAUGAAAGU | UAAUGAAAGU |
| | | G | G |
| | | [SEQ ID | [SEQ ID |
| | | No. 3] | No. 6] |

In some embodiments, the CRISPR-Cas system has sgRNA sequence selected from a group comprising sequences represented by SEQ ID Nos. 1-6 or any combination thereof.

Accordingly, in some embodiments, provided herein is an assay device for determining genetic predisposition to severe forms of SARS-CoV2 infection comprising at least one CRISPR-Cas system having sgRNA sequence selected from a group comprising sequences represented by SEQ ID Nos. 1-6 or any combination

In some embodiments, provided herein is an assay device for determining genetic predisposition to severe forms of SARS-CoV2 infection comprising at least one CRISPR-Cas system having sgRNA sequence selected from a group comprising sequences represented by SEQ ID Nos. 1-6 for genotyping of at least one Single Nucleotide Polymorphism (SNP) selected from a group comprising rs10735079, rs2109069, and rs73064425 or any combination thereof in a biological sample.

In some embodiments, provided herein is an assay device for determining genetic predisposition to severe forms of SARS-CoV2 infection comprising at least one CRISPR-Cas system having sgRNA sequence selected from a group comprising sequences represented by SEQ ID Nos. 1-6 for detection of risk or alternate alleles for at least one Single Nucleotide Polymorphism (SNP) selected from a group comprising rs10735079, rs2109069, and rs73064425 or any combination thereof in a biological sample.

In some embodiments, the CRISPR-Cas system of the assay device has sgRNA sequence selected from a group comprising sequences represented by SEQ ID Nos. 1-3 for detection of risk alleles for at least one Single Nucleotide Polymorphism (SNP) selected from a group comprising rs10735079, rs2109069, and rs73064425 or any combination thereof.

In some embodiments, the CRISPR-Cas system of the assay device has sgRNA sequence selected from a group comprising sequences represented by SEQ ID Nos. 4-6 for detection of alternate alleles for at least one Single Nucleotide Polymorphism (SNP) selected from a group comprising rs10735079, rs2109069, and rs73064425 or any combination thereof.

In some embodiments, the Cas nuclease of the CRISPR-Cas system employed in the present disclosure is selected from a group comprising dCas9, Cas9, Cas-Phi, Cas12, Cas12a, Cas12b, Cas13a or any modifications thereof.

In an exemplary embodiment, the Cas nuclease of the CRISPR-Cas system employed in the present disclosure is Cas12a nuclease.

To enable the aforesaid detection, in some embodiments, the assay device further comprises a reporter system for confirming presence or absence of the SNP(s) of interest in the sample, wherein the reporter system is activated upon recognition of the SNP by the CRISPR guide, by action of the Cas nuclease. Accordingly, in some embodiments, the Cas nuclease that is part of the CRISPR-Cas system performs a dual role of facilitating cleavage at the recognition site, as well as activating the reporter, for the confirmation of a positive event, wherein the positive event indicates the presence of a risk allele or an alternative allele.

In some embodiments, the reporter system produces a detectable signal to indicate a detection event. In some embodiments, reporters employable in the assay device of the present disclosure may provide signals detectable by fluorescence, luminescence, radioactivity, colorimetry, X-ray diffraction or absorption, magnetism, enzymatic activity, and the like.

In some embodiments, the reporter system is selected from but not limited to radioactive labels, enzymes, or chemiluminescent or bioluminescent or fluorescent moieties. In order to effect detection of the analyte/probe hybrid, reporters may be incorporated into reporter reagents comprising a reporter molecule linked to an immuno-reactive or affinity reactive member of a binding pair.

In some embodiments, the reporter system comprises fluorescent reporter molecules such as but not limited to SYT09, fluorescein, rhodamine, rhodoamine600, R-phycoerythrin, and Texas Red.

In some embodiments, the reporter molecule is incorporated in the format of F—(N)ₙ-Q or Q-(N)ₙ—F; wherein F is the fluorescent reporter molecules such as but not limited to SYT09, fluorescein, rhodamine, rhodoamine600, R-phycoerythrin, and Texas Red; wherein N is selected from A, G, T, C, rA, rG, rT and rC; wherein Q is a quencher such as but not limited to black hole quencher (BHQ), Iowa black and 4-(dimethylaminoazo)benzene-4-carboxylic acid (DABCYL) and wherein n ranges from about 6 to about 15.

In embodiments, the substrate/nuclease reaction forms a product which results in a detectable signal, typically a change in color. In many cases, chromogenic substances are an additional requirement for the color reaction. Accordingly, in some embodiments, when the assay device employs enzymes/nucleases as reporters, in order for production of a detectable signal, chromogenic substance(s) may be added to the assay device. Some typical enzyme/chromogen pairs include, but are not limited to; β-galactosidase with chloro-phenol red β-δ-galactopyranoside (CPRG), potassium ferrocyanide or potassium ferricyanide; horse-radish peroxidase with 3,3′ diaminobenzidine (DAB); glucose oxidase with nitro-blue tetrazolium chloride (NBT), alkaline phosphatase with para-nitrophenyl phosphate (PNPP), or 5-bromo-4-chloro-3-indolylphosphate-4-toluidine (BCIP)/NBT.

In some embodiments, the reporter system comprises a reporter pair wherein upon recognition of the target sequence by the guide sequence of the CRISPR-Cas system and hence activation of activity of the Cas nuclease, members of the reporter pair are designed to be separated by the Cas nuclease, resulting in signal modification that can then be detected. Therefore, in order to facilitate the detection of the presence or absence of SNPs in the sample, in some embodiments. also immobilized on to the assay device (in any of the aforesaid formats) is the reporter system.

In exemplary embodiments, the Cas nuclease of the CRISPR-Cas system employed in the present disclosure is Cas12a. Without intending to be limited by theory, Cas12a nuclease binds to a guide RNA to make Cas12a:guide RNA complex. The Cas12a:guide RNA complex, in the presence of target DNA, makes a trimeric nucleoprotein complex, Cas12a:guide RNA:target DNA. This trimeric nucleoprotein complex has an endonuclease activity and it cleaves ssDNA irrespective of its sequence. If the termini of a ssDNA are labeled with a fluorophore and quencher pair, its cleavage by Cas12a:guideRNA:target DNA complex leads to an increase in fluorescence. Such an ssDNA may be used as a reporter to detect the presence of Cas12a:guide RNA:target DNA or indirectly target DNA.

Accordingly, in some embodiments, reagents in the assay device comprise Cas12a enzyme, guide RNA selected from a group comprising sequences represented by SEQ ID Nos. 1-6, and a reporter such as an ssDNA labeled with a fluorophore and quencher pair. If the sample to be detected contains the target DNA; the Cas12a:guide RNA:target DNA will form and will cleave the ssDNA that will result in an increase in fluorescence signal. Said embodiments of the present disclosure therefore make us of the trans-cleavage mechanism of the Cas12a nuclease.

As per the known mechanism of the Cas nuclease, a protospacer adjacent motif (PAM) is required for a Cas nuclease to perform cleavage and is generally found 3-4 nucleotides downstream from the cut site.

In some embodiments, when the target locus does not naturally contain a PAM sequence, the target locus is converted to a Cas susceptible site by artificially introducing a PAM sequence. In some embodiments, the PAM sequence is artificially introduced by using primer(s) designed to comprise the PAM sequence, for amplification of the target loci; wherein the amplification of the target loci takes place within the device or prior to application to the device. Sequence(s) amplified using said primers therefore comprise the PAM sequence.

In exemplary embodiments of the present disclosure, the assay device is designed to determine genetic predisposition to severe forms of COVID-19 infection. Said detection, in a non-limiting embodiment is performed on a biological sample, wherein the biological sample comprises nucleic acid. In some embodiments, the sample applied to the detection device characterized by the aforesaid features is an unprocessed sample or a pre-processed sample. An unprocessed sample is any biological sample which can act as source of genetic material such as but not limited to blood, urine, feces, sperm, saliva, tissue biopsy and intraoral mucosa. Examples of pre-processed samples include but are not limited to nucleic acids isolated and amplified from biological samples. In preferred embodiments, the nucleic acids isolated from the sample are amplified at the regions of the SNP(s) i.e. the target loci. In some embodiments, the amplification is achieved by methods such as but not limited to Polymerase Chain Reaction (PCR), recombinase polymerase amplification (RPA), strand displacement amplification (SDA), ligase chain reaction (LCR), Nucleic Acid Sequence Based Amplification (NASBA), transcription-associated amplification (TAA), Cold PCR, and Non-Enzymatic Amplification Technology (NEAT).

In an exemplary embodiment, the amplification is achieved by Polymerase Chain Reaction PCR.

In a non-limiting embodiment, the primers employed for amplification are targeted towards the loci of the SNP(s) of interest.

In some embodiments, the assay device is designed such that nucleic acid isolation and amplification occurs within the assay device. Alternatively, in some embodiments, the sample is partially processed so as to isolate the nucleic acids, such that said nucleic acids are applied to the assay device and the amplification at target loci occurs within the assay device. Accordingly, in some embodiments, the assay device may further comprise reagents to facilitate the isolation of nucleic acids from the unprocessed sample and/or amplification at target loci. Said reagents that facilitate the isolation of nucleic acids from the unprocessed sample and/or amplification at target loci include but are not limited to enzyme(s), dNTPs, buffer(s) and primer(s).

In a non-limiting embodiment, the enzyme for facilitating amplification is a polymerase enzyme. With regard to the primer(s), the primer(s) are designed as per the sequence at the target loci or template. The primers are designed such that minimal amount of non-specific amplification is detected and further, such that amplification of multiple amplicons is facilitated through a minimum number of reactions. In some embodiments, as explained above, the primers (employed as part of pre-processing of the sample or for amplification on the assay device) are designed to comprise the PAM sequence to yield Cas susceptible amplified nucleic acids.

Accordingly, in order to facilitate said detection and allow action of the Cas nuclease by CRISPR-Cas systems employing guide sequences as described in Table, primer sequences employed for amplification of the SNP loci in the present disclosure include but are not limited to—

TABLE C

| SNP ID | Forward primer | Reverse primer |
|---|---|---|
| rs10735079 | CGTGTATTACGA | AAGATGCTTGG |
| | AGGAGAACACAA | CCCTCCCACAG |
| | [SEQ ID No. 7] | [SEQ ID No. 8] |
| rs2109069 | GGGTTTTGGGCTC | GTGAGGTTGAA |
| | CAAGAGATTTGGG | TGGCCTGTCC |
| | [SEQ ID No. 9] | [SEQ ID No. 10] |
| rs73064425 | TGAATGTGGCCTA | ACAGTGGCTGG |
| | TGCTGGT | TCATCATAC |
| | [SEQ ID No. 11] | [SEQ ID No. 12] |

In some embodiments, the reactions in the assay device that enable genotyping of the above defined SNPs are optimized by controlling parameters such as temperature, pH, duration of assay, template concentration and salt composition.

In some embodiments, the assay device disclosed herein is prepared in freeze-dried format for convenient distribution. However, when put to application, the assay device performs the assay at ambient temperature, preferably ranging from about 25° C. to about 37° C.

In some embodiments, the assay device optionally comprises attachments such as electrodes for introducing electric field or electrically or battery controlled means of regulating temperature to optimize the performance of the assay device when put to application at the point of care.

In some embodiments, the assay device further comprises reaction buffer(s) that facilitates action of the Cas nuclease. In some embodiments, the reaction buffer comprises components such as but not limited to buffering agent(s) such as but not limited to Tris buffer, salt(s) such as but not limited to NaCl, and stabilizer(s) such as but not limited to BSA.

In some embodiments, the buffering agent is selected from but not limited to Tris buffer, Phosphate Buffer Saline (PBS), HEPES and Citrate. In some embodiments, pH of the reaction buffer ranges from about 6.0 to about 8.0.

In some embodiments, the salt is selected from a group comprising potassium chloride (KCl), sodium chloride (NaCl), magnesium chloride (MgCl₂) and Ethylenediaminetetraacetic acid (EDTA) or combinations thereof. In some embodiments, the reaction buffer comprises KCl at a concentration of about 0 mM to about 150 mM, NaCl at a concentration of about 0 mM to about 150 mM, and/or EDTA at a concentration of about 0 mM to about 1 mM.

In some embodiments, the stabilizer is selected from a group comprising heparin and bovine serum albumin (BSA) or a combination thereof. In some embodiments, the reaction buffer comprises heparin at a concentration of about 0 μg/mL to about 50 μg/mL, and/or BSA at a concentration of about 0 μg/mL to about 100 μg/mL.

In some embodiments, the reaction buffer may comprise additional reagents to optimize the detection reaction in the assay device.

In some embodiments, the reaction buffer is already present in the wells, depressions or containers of the assay device before the sample is applied or is added post application of the sample to the assay device. In some embodiments. the reaction buffer is the same buffer employed for amplification of the target loci.

Accordingly, in some embodiments, the reaction buffer is introduced into the assay device along with the sample as part of an amplification mix comprising the amplified target loci.

The present disclosure further provides a method for genotyping of SNP(s) selected from a group comprising rs10735079, rs2109069 and rs73064425 or any combination thereof in a sample, wherein said method comprises—
applying the sample to the assay device described above to obtain a detectable signal.

In some embodiments, the sample is a freshly collected sample or a sample previously collected at another location.

In some embodiments, the sample is an unprocessed sample or a pre-processed sample.

Examples of unprocessed samples include but are not limited to blood, urine, feces, sperm, saliva, tissue biopsy and intraoral mucosa. Alternatively, the sample employed in the said assay is a pre-processed sample such as nucleic acids isolated and amplified from biological samples. The assay and the method employing said assay as described herein can detect both DNA and RNA with comparable levels of sensitivity and can differentiate targets from non-targets based on single base pair differences. In preferred embodiments, the nucleic acid isolated and amplified from biological samples is DNA.

In preferred embodiments, the nucleic acids isolated from the sample are amplified at the regions of the SNP(s) i.e. the target loci.

In some embodiments, the target loci are amplified through a single reaction or multiple reactions. In exemplary embodiments, the target loci for all SNPs to be detected in the sample are amplified through a single reaction.

In exemplary embodiments, the sample is a blood sample, wherein the blood sample is processed to extract/isolate DNA and subjected to an amplification reaction to amplify the target loci.

In some embodiments, the extraction or isolation of nucleic acids is performed by any method practiced in the art.

In some embodiments, the amplification is by methods such as but not limited to polymerase chain reaction (PCR), recombinase polymerase amplification (RPA), strand displacement amplification (SDA), ligase chain reaction (LCR), Nucleic Acid Sequence Based Amplification (NASBA), transcription-associated amplification (TAA), Cold PCR, and Non-Enzymatic Amplification Technology (NEAT).

In some embodiments, the isolation and amplification are performed in two separate steps. In some embodiments, the isolation and amplification are performed in a single step reaction.

In some embodiments, the isolation and amplification are performed prior to application of the sample to the device. In some embodiments, the isolation is performed prior to application of the sample to the device and the amplification occurs on the assay device. In some embodiments, the isolation and the amplification occur on the assay device, preferably when the sample is applied in an unprocessed form.

Accordingly, in some embodiments, the present disclosure provides a method for genotyping of SNP(s) selected from a group comprising rs10735079, rs2109069 and rs73064425 or any combination thereof in a sample, wherein said method comprises—
extracting nucleic acid from the sample;
applying the extracted nucleic acid to the assay device described above to obtain a detectable signal.

In some embodiments, the present disclosure provides a method for detection of risk or alternate alleles for SNP(s) selected from a group comprising rs10735079, rs2109069 and rs73064425 or any combination thereof in a sample, wherein said method comprises—
extracting nucleic acid from the sample;
amplifying target loci in the extracted nucleic acid;
applying the amplified target loci to the assay device described above to obtain a detectable signal.

In some embodiments, the present disclosure provides a method for detection of risk or alternate alleles for SNP(s) selected from a group comprising rs10735079, rs2109069 and rs73064425 or any combination thereof in a sample, wherein said method comprises—
extracting nucleic acid from the sample;
amplifying target loci in the extracted nucleic acid using primer(s) selected from a group of sequences represented by SEQ ID Nos. 7-12; and
applying the amplified target loci to the assay device described above comprising CRISPR-Cas system having sgRNA(s) selected from a group of sequences represented by SEQ ID Nos. 1-6; to obtain detectable signal.

In some embodiments, the amplification is performed on the device, within the wells. In said scenario, the assay device further comprises reagents to facilitate amplification of nucleic acids, either directly from the unprocessed sample or from nucleic acids isolated from the sample prior to application on the assay device. By amplifying the target sequences, the sensitivity of the assay is improved, since fewer target sequences are needed at the beginning of the assay to better ensure detection of the target SNP in the sample.

When the amplification is performed prior to application of the sample to the device, the sample applied to the device is in the form of the amplified nucleic acid or an amplification mix from the amplification reaction. In some embodiments, the amplification mix comprises the amplified nucleic acids/amplified target loci and the reagents that facilitated the amplification reaction such as but not limited to template(s), buffer(s), forward/reverse primer(s). In some embodiments, the forward/reverse primer(s) designed for amplification are designed to comprise PAM sequence(s) so that their binding to the amplified nucleic acids converts the target site into a site susceptible to cleavage by the Cas nuclease of the CRISPR-Cas system in the assay device.

Accordingly, in some embodiments, the present disclosure provides a method for genotyping of SNP(s) selected from a group comprising rs10735079, rs2109069 and rs73064425 or any combination thereof in a sample, wherein said method comprises—
extracting nucleic acid from the sample;
amplifying target loci in the extracted nucleic acid to obtain an amplification mix comprising the amplified nucleic acid;
applying the amplification mix nucleic acid to the assay device described above; and
observing a detectable signal.

In some embodiments, the present disclosure provides a method for genotyping of SNP(s) selected from a group comprising rs10735079, rs2109069 and rs73064425 or any combination thereof in a sample, wherein said method comprises—
extracting nucleic acid from the sample;
amplifying target loci in the extracted nucleic acid to obtain an amplification mix comprising the amplified nucleic acid using primer(s) selected from a group of sequences represented by SEQ ID Nos. 7-12;
applying the amplification mix nucleic acid to the assay device described above comprising CRISPR-Cas system having sgRNA(s) selected from a group of sequences represented by SEQ ID Nos. 1-6, and
observing a detectable signal.

The detectable signal in the aforesaid method is observed by virtue of the CRISPR-Cas system recognizing and cleaving the target (risk allele and/or alternative allele). In some embodiments, said recognition of the target allele is accompanied by simultaneous activation of the reporter system by the Cas nuclease, yielding a detectable (preferably quantifiable) signal. In exemplary embodiments, the detectable signal is fluorescence or a colorimetric reaction.

In some embodiments, the detectable signal is observable by the naked eye. Such an observation provides an indication of the presence or absence of the risk allele and/or the alternative allele. For quantification, in some embodiments, the signal is measured using a spectrophotometer, a colorimeter, a fluorometer or a luminometer depending on the reporter system employed.

In some embodiments, the method envisaged in each of the above embodiments is an in-vitro method.

The present disclosure therefore provides an in-vitro method for genotyping of SNP selected from a group comprising rs10735079, rs2109069 and rs73064425 or any combination thereof in a sample, wherein said method comprises—
applying the sample to the assay device described above to obtain a detectable signal.

In some embodiments, the present disclosure provides an in-vitro method for detection of risk or alternate alleles for SNP(s) selected from a group comprising rs10735079, rs2109069 and rs73064425 or any combination thereof in a sample, wherein said method comprises—
extracting nucleic acid from the sample; and
applying the extracted nucleic acid to the assay device described above to obtain a detectable signal.

In some embodiments, provided herein is an in-vitro method for genotyping of SNP selected from a group comprising rs10735079, rs2109069 and rs73064425 or any combination thereof in a sample, wherein said method comprises—
extracting nucleic acid from the sample;
amplifying target loci in the extracted nucleic acid using primer(s) selected from a group of sequences represented by SEQ ID Nos. 7-12; and
applying the amplified target loci to the assay device described above comprising CRISPR-Cas system having sgRNA(s) selected from a group of sequences represented by SEQ ID Nos. 1-6; to obtain detectable signal.

In some embodiments, provided herein is an in-vitro method for detection of risk or alternate alleles for SNP(s) selected from a group comprising rs10735079, rs2109069 and rs73064425 or any combination thereof in a sample, wherein said method comprises—
extracting nucleic acid from the sample;
amplifying target loci in the extracted nucleic acid to obtain an amplification mix comprising the amplified target loci; and
applying the amplification mix nucleic acid to the assay device described above to obtain a detectable signal.

In some embodiments, the in-vitro method for detection of risk or alternate alleles for SNP(s) selected from a group comprising rs10735079, rs2109069 and rs73064425 or any combination thereof in a sample, comprises—
extracting nucleic acid from the sample;
amplifying target loci in the extracted nucleic acid to obtain an amplification mix comprising the amplified nucleic acid using primer(s) selected from a group of sequences represented by SEQ ID Nos. 7-12;
applying the amplification mix nucleic acid to the assay device described above comprising CRISPR-Cas system having sgRNA(s) selected from a group of sequences represented by SEQ ID Nos. 1-6, to obtain a detectable signal.

In some embodiments, the aforesaid method is performed at ambient temperature. In a non-limiting embodiment, the aforesaid method is performed at a temperature ranging from about 25° C. to about 37° C.

In some embodiments, the aforesaid method is performed at the point of sample collection (point of care) or in a separate setting from the point of sample collection.

In exemplary embodiments, the aforesaid method is performed at the point of care.

In some embodiments, the aforesaid method provides a detectable signal or readout in about 10 minutes to about 120 minutes after application of the amplified nucleic acid or the amplification mix.

In some embodiments, the detectable signal is observed as a qualitative reading merely confirming presence or absence of an SNP or a risk allele in a sample. In some embodiments, the detectable signal is measured using a spectrophotometer, a colorimeter, a fluorometer or a luminometer depending on the reporter system employed. The readings from said device(s) may then be manually compared with pre-determined standard values.

Alternatively, the detectable signal is fed into an automated system, wherein an algorithm compares the detectable signal with pre-fed standard values to determine the extent of predisposition of an individual to severe forms of SARS-CoV2 infection. Said automated system therefore helps categorize the individual as a high-risk or low-risk candidate.

In some embodiments, the assay device and method of the present disclosure provide a risk analysis based on correlation of specific SNPs to severe forms of SARS-CoV2 infection by referencing genotype data for the polymorphic marker (SNP) to a database or an automated system that comprises data establishing correlations between at least one allele of the polymorphism and severe forms of the infection. In some embodiments, the database or automated system comprises a correlation for one polymorphism. In other embodiments, the database automated system comprises a correlation for a plurality of polymorphisms pertinent to severe SARS-CoV2 infection. In some embodiments, the correlation is reported as a statistical measure. Accordingly, in some embodiments, when the database or automated system receives the detectable signal from the assay device, the database or automated system provides a risk analysis based on the established correlation.

In some embodiments, genetic predisposition to severe forms of SARS-CoV2 infection is determined by presence or absence of SNPs selected from a group comprising rs10735079, rs2109069 and rs73064425 or any combination thereof in the biological sample.

Accordingly, in some embodiments, the present disclosure provides a method for detecting predisposition to severe forms of SARS-CoV2 infection, wherein said method comprises—
applying the sample to the assay device described above to obtain a detectable signal.

In some embodiments, the aforesaid method allows point of care testing for detection of genetic predisposition to severe forms of SARS-CoV2 infection and/or determining the extent of such predisposition

Since the assay device of the present disclosure and the method(s) described above each rely heavily upon the detection of the SNPs of interest by specifically designed guide sequences of the CRISPR-Cas system, the present disclosure further provides specific CRISPR guide sequences designed to detect the SNPs as set out in Table A.

In some embodiments, the CRISPR guides (sgRNAs) are designed to target the risk allele or the alternative allele of said SNPs.

More specifically, provided herein is sgRNA sequence against loci of SNP(s) selected from a group comprising rs10735079, rs2109069 and rs73064425 or any combination thereof.

In some embodiments, the sgRNA has sequence selected from a group comprising sequences represented by SEQ ID Nos. 1-6.

In some embodiments, envisaged herein is a combination of one or more sgRNA sequences selected from a group comprising sequences represented by SEQ ID Nos. 1-3.

In some embodiments, envisaged herein is a combination of one or more sgRNA sequences selected from a group comprising sequences represented by SEQ ID Nos. 4-6.

Also envisaged in the scope of the present disclosure are modifications to the sequences disclosed in Table B to optimize the recognition of target loci by the CRISPR-Cas system. In some embodiments, said modifications include but are not limited to modification of length and introduction of additional mismatches.

Further envisaged in the present disclosure is the sgRNA sequence(s) as defined above for use in detecting genetic predisposition to severe forms of SARS-CoV2.

Further provided herein is CRISPR-Cas system(s) comprising specific CRISPR guide sequence(s) as defined above. In some embodiments, the Cas nuclease of the CRISPR-Cas system is selected from a group comprising dCas9, Cas9, Cas-Phi, Cas12, Cas12a, Cas12b, Cas13 or any modifications thereof. In an exemplary embodiment, the Cas nuclease of the CRISPR-Cas system is Cas12a.

In some embodiments, provided herein is CRISPR-Cas system(s) comprising specific CRISPR guide sequence(s) as defined above. In some embodiments, the Cas nuclease of the CRISPR-Cas system is selected from a group comprising dCas9, Cas9, Cas-Phi, Cas12, Cas12a, Cas12b, Cas13 or any modifications thereof. In an exemplary embodiment, the Cas nuclease of the CRISPR-Cas system is Cas12a.

In an exemplary embodiment. the CRISPR-Cas system(s) comprises sgRNA sequence(s) selected from a group comprising sequences represented by SEQ ID Nos. 1-6 in conjunction with Cas12a nuclease.

Cas nuclease Cas nuclease In some embodiments, the present disclosure further relates to use of a CRISPR-Cas system as defined above for detection of SNPs selected from a group comprising rs10735079, rs2109069 and rs73064425 or any combination thereof in a sample, wherein presence of said SNPs is indicative of genetic predisposition to severe forms of SARS-CoV2.

In some embodiments, the features defined for the aforesaid assay and the method(s) are applicable to the use as mentioned above.

The present disclosure further provides a kit comprising the assay device as defined above or the sgRNA sequences as defined above, along with an instruction manual.

In some embodiments, the kit further comprises one or more component(s) selected from a group comprising a multi-well plate, reaction buffer(s), means for sample procurement, means for application of the sample to the assay device, primer(s), dNTPs, polymerase enzyme(s) and reporter system(s) or any combination thereof.

In a non-limiting embodiment, examples of the means for sample procurement include but are not limited to syringe, nasal swab, throat swab and sample container. In a non-limiting embodiment, examples of the means for sample application include but are not limited to syringe, dropper and pipette.

Each of said components are defined in earlier embodiments in the context of the assay device or related method(s).

In some embodiments, instead of a pre-prepared assay device, the above described kit of the present disclosure may comprise the CRISPR guide sequence or the CRISPR-Cas system of the present disclosure along with the a patterned substrate, set of beads or reaction tubes, along components selected from a group comprising reaction buffer(s), Cas nuclease(s), means to immobilize the CRISPR-Cas system, means to procure a sample, means to apply the sample to the assay device, primer(s), dNTPs, polymerase enzyme(s), reporter system(s), instructions manual or any combination thereof.

The subject matter of embodiments of the present invention is described here with specificity to meet statutory requirements, but this description is not necessarily intended to limit the scope of the disclosure. The disclosed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

It is to be understood that the foregoing descriptive matter is illustrative of the disclosure and not a limitation. While considerable emphasis has been placed herein on the particular features of this disclosure, it will be appreciated that various modifications can be made, and that many changes can be made in the preferred embodiments without departing from the principles of the disclosure. Those skilled in the art will recognize that the embodiments herein can be practiced with modification within the spirit and scope of the embodiments as described herein. Similarly, additional embodiments and features of the present disclosure will be apparent to one of ordinary skill in art based upon description provided herein.

Descriptions of well-known/conventional methods/steps and techniques are omitted so as to not unnecessarily obscure the embodiments herein. Further, the disclosure herein provides for examples illustrating the above-described embodiments, and in order to illustrate the embodiments of the present disclosure certain aspects have been employed. The examples used herein for such illustration are intended merely to facilitate an understanding of ways in which the embodiments herein may be practiced and to further enable those of skill in the art to practice the embodiments herein. Accordingly, the following examples should not be construed as limiting the scope of the embodiments herein.

### EXAMPLES

### Example 1: Prototype Validation on Synthetic Targets

In order to build a prototype of the assay of the present disclosure, a series of guide RNA pairs were designed for each SNP corresponding alterative allele and risk allele for each SNP an alternative allele specific guide and a risk allele specific guide was designed (Table B). Selectivity of various such guide pairs for the variant detection was initially tested on the synthetic targets for each SNP. The synthetic targets for each SNP corresponding to each allele, alternative and risk, were prepared by overlap extension of the two partially complementary oligonucleotides.

### Overlap Extension

An overlap extension mix was prepared by mixing overlapping oligonucleotides at a concentration of about 1 μM in 1×PCR master mix and overlap extension was performed in thermal cyclers with following conditions: Initial heat denaturation at about 95° C. for about 5 minutes; 15 cycles of heat denaturation at about 90° C. for about 30 seconds, annealing at about 60° C. for about 30 seconds, extension at about 72° C. for about 30 seconds; and final extension for about 5 minutes at about 72° C. The products obtained after overlap extension and one step RT-PCR were purified using a purification kit and quantified based on absorbance at about 260 nm in NanoDrop One™ (Thermo Scientific, USA).

The guide selectivity for each SNP pair using the prepared synthetic targets was studied through trans-cleavage assays.

### Trans-Cleavage Assay

Trans-cleavage assays for a given target template or sample was carried out using about 25 nM of LbaCas12a, about 25 nM of the indicated guide RNA in a solution containing about 50 mM NaCl, about 10 mM Tris-HCl, about 10 mM MgCl2, and about 100 μg/ml BSA at a pH of about 7.9, at a temperature of about 25° C., at about 37° C. All the reactions were initiated by addition of the synthetic target or PCR amplified target from genomic DNA, and it was ensured that the time difference between the beginning of addition of the sample and the start of the data acquisition was not more than about 2 minutes.

The trans-cleavage assays were done in the presence of the synthetic targets corresponding to both the variants of the SNP i.e. risk allele and alternate allele. A good guide RNA for alternative allele is expected to show faster trans-cleavage in the presence of the alternative allele than in the presence of risk allele, variant and vice versa.

A quantitative criterion was set up for selecting a good guide RNA as described below. A value referred to as “rate ratio” was determined for each guide. Rate ratio indicates, approximately, the relative rate of trans-cleavage by a LbaCas12a:guide RNA complex, in presence of the allele it is expected to detect compared to the one it is supposed to discriminate against. The following formula was devised for calculation of rate ratio:

Rate ratio for alternative guide=(Intensity of detectable signal in the presence of the alternative allele at 30 minutes from the start of data acquisition−Intensity of detectable signal in the presence of the alternative allele at 10 minutes from the start of data acquisition)/(Intensity of detectable signal in the presence of risk allele at 30 minutes from the start of data acquisition−intensity of detectable signal in the presence of risk allele at 10 minutes from the start of data acquisition).

Rate ratio for risk guide=(Intensity of detectable signal in the presence of the risk allele at 30 minutes−Intensity of detectable signal in the presence of the risk allele at 10 minutes from the start of data acquisition)/(Intensity of detectable signal in the presence of alternative allele at 30 minutes from the start of data acquisition-intensity of detectable signal in the presence of alternative allele at 10 minutes from the start of data acquisition).

A good, selective guide RNA (sgRNA) was expected to have a rate ratio of greater than 2 at 5 nM target concentration under the assay conditions employed. Using this criteria, selective guide pair for the SNPs defined in Table A. Table A shows the sgRNA that qualified this criterion.

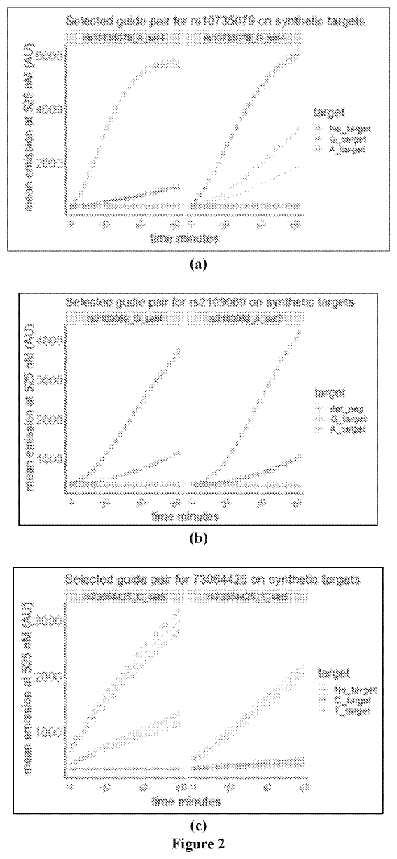

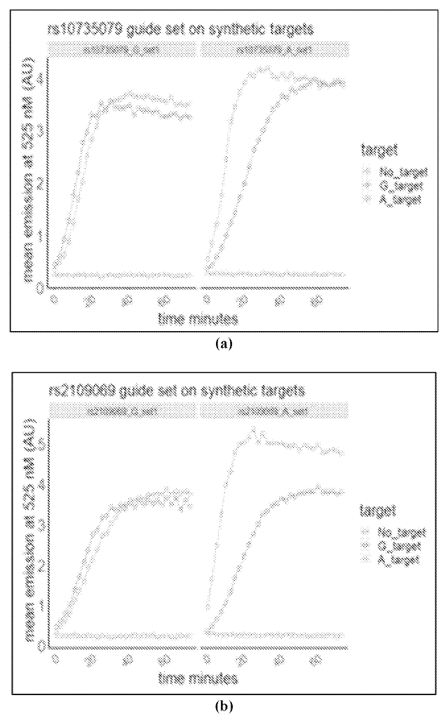

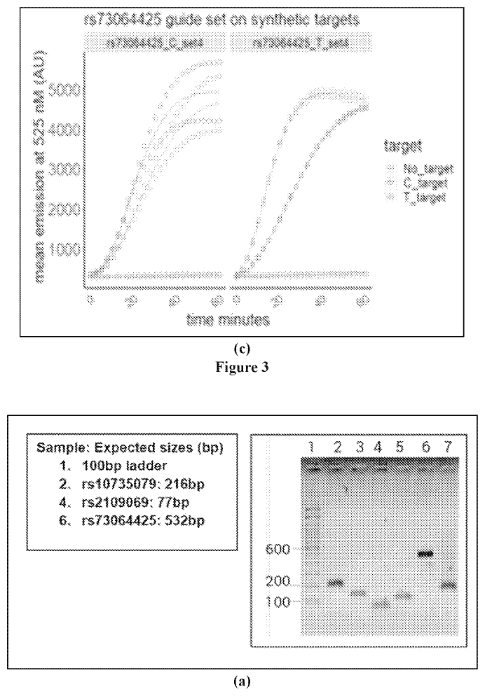

For the purposes of exemplification, the data for the trans-cleavage assay using synthetic target for the detection of SNPs rs10735079, rs2109069, and rs73064425 is provided herein. FIG. 2 depicts the results of the trans-cleavage assay on the synthetic targets for detection of the three SNPs as defined above for which variant selectivity was observed a) the guide pair for rs10735079 b) the guide pair for rs2109069, and d) the guide pair for rs73064425.

FIG. 2a shows the trans-cleavage data for the guide pair selected for SNP rs10735079. The guide pair was designed to be selective for variants A (risk allele) and G (alternative allele). The left panel shows the trans-cleavage rates of the CRISPR-Cas complex containing variant A selective guide in the presence of synthetic targets. The variant A selective guide showed faster trans-cleavage in the presence of the variant A synthetic target (risk allele) than in the presence of variant G synthetic target (alternate allele). In the presence of 5 nM of synthetic targets, the rate ratio of the variant A selective guide was 12 (FIG. 2a left panel). The right panel shows the trans-cleavage rates of the CRISPR-Cas complex containing variant G selective guide in the presence of synthetic targets. The variant G selective guide showed faster trans-cleavage in the presence of the variant G synthetic target (alternate allele) than in the presence of variant A synthetic target (risk allele). In the presence of 5 nM of synthetic targets, the rate ratio of the variant G selective guide was 3 (FIG. 2a).

FIG. 2(b) shows the trans-cleavage data for the guide pair selected for SNP rs2109069. The guide pair was designed to be selective for variants G (alternate allele) and A (risk allele) The left panel shows the trans-cleavage rates of guide selective for the variant G in the presence of synthetic targets. The variant G selective guide showed faster trans-cleavage in the presence of the variant G synthetic target (alternative allele) than in the presence of the variant A synthetic target (risk allele). In the presence of 5 nM of synthetic targets the rate ratio was 5.3 (FIG. 2c left panel). The right panel shows the trans-cleavage rates of the CRISPR-Cas complex containing variant G selective guide in the presence of synthetic targets. The variant A selective guide showed faster trans-cleavage in the presence of the variant A synthetic target (risk allele) than in the presence of variant G synthetic target (alternative allele). In the presence of 5 nM of synthetic targets the rate ratio was 7.6 (FIG. 2c right panel).

FIG. 2(c) shows the trans-cleavage data for the guide pair selected for SNP rs73064425. The guide pair was designed to be selective for variants C (alternate allele) and T (risk allele). The left panel shows the trans-cleavage rates of guide selective for the variant C in the presence of synthetic targets. The variant C selective guide showed faster trans-cleavage in the presence of the variant C synthetic target (alternate allele) than in the presence of variant T synthetic target (risk allele). In the presence of 5 nM of synthetic targets, the rate ratio was 2.6 (FIG. 2(c) left panel). The right panel shows the trans-cleavage rates of the CRISPR-Cas complex containing variant T selective guide in the presence of synthetic targets. The variant T selective guide showed faster trans-cleavage in the presence of the variant T synthetic target (risk allele) than in the presence of variant C synthetic target (alternate allele). In the presence of 5 nM of synthetic targets, the rate ratio was 9.5 (FIG. 2(c) right panel).

Based on the above, the prototype using the guides of the present disclosure was validated on synthetic targets and selectivity of the guides was confirmed.

### Example 2: Guide Design—Comparative Example

As mentioned above, the guides reported in Example 1 were arrived at after testing multiple guide sequences against each SNP described in Table A. The advantages of the specific guide sequences described in Table B and exemplified in Example 1 over others designed against the same target are derivable from FIG. 3. The figure is representative of the reduction in guide selectivity observed upon deviation from the guide design of the present disclosure. Alternative guides employed for the purposes of comparison are provided below—

TABLE D

| | | Comparative | Comparative |
|---|---|---|---|
| | | guide | guide |
| | | sequence | sequence |
| S. | | [risk | [alternate |
| No. | SNP | allele] | allele] |
| 1. | rs10735079 | UAAUUUCUAC | UAAUUUCUA |
| | | UAAGUGUAGA | CUAAGUGUA |
| | | UGUUUCGUCC | GAUGUUUUG |
| | | CCAGGCCCCU | UCCCCAGGC |
| | | A | CCCUA |
| | | [SEQ ID | [SEQ ID |
| | | No. 13] | No. 14] |
| 2. | rs2109069 | UAAUUUCUAC | UAAUUUCUA |
| | | UAAGUGUAG | CUAAGUGUA |
| | | AUGGCUCCA | GAUGGUUCC |
| | | CUCCCCUUC | ACUCCCCUU |
| | | CUCU | CCUCU |
| | | [SEQ ID | [SEQ ID |
| | | No. 15] | No. 16] |
| 3. | rs73064425 | UAAUUUCUA | UAAUUUCUA |
| | | CUAAGUGUA | CUAAGUGUA |
| | | GAUACGAAU | GAUAUGAAU |
| | | UAAUGAAAG | UAAUGAAAG |
| | | UGGAU | UGGAU |
| | | [SEQ ID | [SEQ ID |
| | | No. 17] | No. 18] |

The above guides targeting the same region as the guides of the present disclosure were employed for a trans-cleavage assay on the synthetic targets for the three SNPs. Results of the said trans-cleavage assay are depicted in FIG. 3.

FIG. 3(a) shows the trans-cleavage data for the comparative guide pair selected for SNP rs10735079. The guide pair was designed to be selective for variants G (alternative) and A (risk). The left panel shows the trans-cleavage rates of the CRISPR-Cas complex containing variant G selective guide in the presence of synthetic targets. The variant G selective guide showed similar rates of trans-cleavage in the presence of synthetic target of both the variants (risk allele as well as alternate allele). In the presence of 5 nM of synthetic targets the rate ratio was 0.77; and hence based on the criteria set, it was determined that the guide was not-selective (FIG. 3(a) left panel). The right panel shows the trans-cleavage rates of the CRISPR-Cas complex containing variant A selective guide in the presence of synthetic targets. The variant A selective guide showed faster trans-cleavage in the presence of the variant A synthetic target as compared to the variant G synthetic target. However, the rate ratio was calculated to be 0.84; and hence was determined as being not-selective guide (FIG. 3(a) right panel).

FIG. 3(b) shows the trans-cleavage data for the guide pair selected for SNP rs2109069. The guide pair was designed to be selective for variants G (alternate allele) and A (risk allele). The left panel shows the trans-cleavage rates of the CRISPR-Cas complex containing variant G selective guide in the presence of synthetic targets. It shows similar trans-cleavage rates in the presence of both variants of the synthetic target. In the presence of 5 nM of synthetic targets the rate ratio was 0.77; and hence it was determined as being not-selective (FIG. 3b left panel) The right panel shows the trans-cleavage rates of the CRISPR-Cas complex containing variant A selective guide. It showed similar trans-cleavage rates in the presence of both variants of the synthetic target (risk allele and alternate allele). In the presence of 5 nM of synthetic targets the rate ratio was 0.84; and hence it was determined as being not selective (FIG. 3(b) right panel).

FIG. 3(c) shows the trans-cleavage data for the guide pair selected for SNP rs73064425. The guide pair was designed to be selective for variants C and T. The left panel shows the trans-cleavage rates of the CRISPR-Cas complex containing variant C selective guide in the presence of synthetic targets. It showed similar trans-cleavage rates in the presence of both variants of the synthetic target (risk allele and alternate allele). In the presence of 5 nM of synthetic targets the rate ratio was 1.3; and hence it was determined as being not selective (FIG. 3(c) left panel). The right panel shows the trans-cleavage rates of the CRISPR-Cas complex containing variant T selective guide. It showed similar trans-cleavage rates in the presence of both variants of the synthetic target. In the presence of 5 nM of synthetic targets, the rate ratio was 1.4; and hence it was determined as being not-selective (FIG. 3(c) right panel).

The results of the above assay in comparison with the results of Example 1 are clearly depictive of the merit of the guide sequences of the present disclosure. The guides of the present disclosure are able to showcase high selectivity to render a conclusive result on presence or absence of the SNPs of interest [rs10735079, rs2109069, and rs73064425] as opposed to the alternative guides (representative examples provided above) that are unable to exhibit such selectivity.

### Example 3: Primer Design for DNA Amplification

A series of primer sequences were scanned for each SNP of interest [rs10735079, rs2109069, and rs73064425].

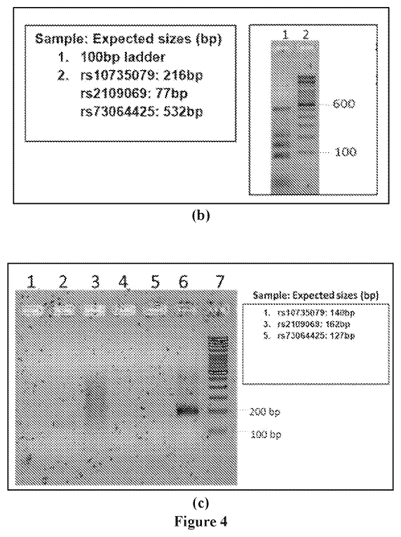

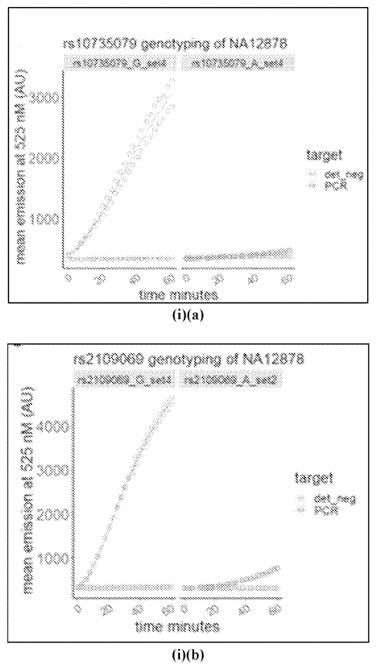

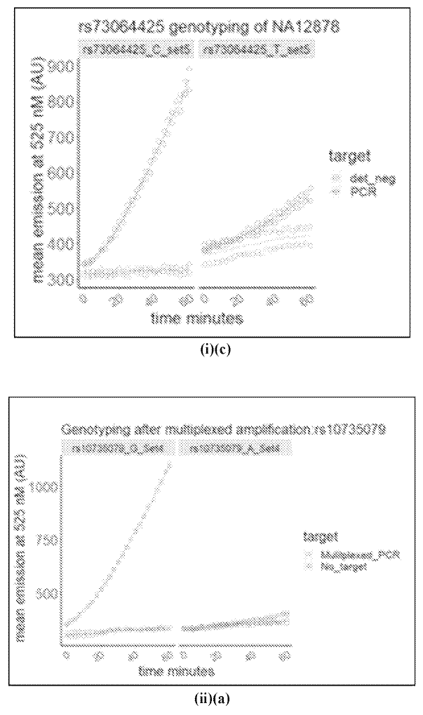

Based on the said screening, a primer set that yielded the amplicon of the intended length for each SNP was shortlisted (Table C). FIG. 4(a) shows the gel electrophoresis results of an amplicon of the expected size.

FIG. 4(a) depicts a gel electrophoresis image showing the amplification of the products of expected sizes using NA12878 genomic DNA using the primers of Table C. FIG. 4(a) shows amplification of SNP in different tubes and (b) shows multiplexed amplification of rs10735079, rs2109069, and rs73064425.

FIG. 4(a) Lane 1 shows the 100 bp ladder marker. Lane-1 has PCR product amplified using the primer set designed for the amplification of regions of the genomic DNA that harbors SNP rs10735079. The primer sets were designed such that the length of the amplified product was 216 bp. The fact that the band in Lane 2 is slightly above 200 bp confirms that it is the correct product, and absence of any other band indicates that non-specific amplicons were not present at detectable levels. Lane-4 shows PCR product amplified using the primer set designed for the amplification of regions of the genomic DNA that harbors SNP rs2109069. The primer sets were designed such that the length of the amplified product was 77 bp. The fact that the band in Lane 4 is slightly below 100 bp confirms that it is the correct product, and absence of any other band indicates that non-specific amplicons were not present at detectable levels. Lane-6 has PCR product amplified using the primer set designed for the amplification of regions of the genomic DNA that harbors SNP rs73064425. The primer sets were designed such that the length of the amplified product was 532 bp. The fact that the band in Lane 6 is slightly below 600 bp confirms that it is the correct product, and absence of any other band indicates that non-specific amplicons were not present at detectable levels.

FIG. 4(b) shows the multiplexed amplification of the SNPs using the primers of Table C. Lane-1 is a 100 bp ladder. Lane-2 shows amplification done in a single tube using the primer set for the following SNPs: f rs10735079, rs2109069, and rs73064425. The expected length of rs10735079 was 216 bp and the band slightly above 200 bp is the band corresponding to it. The expected length of rs2109069 was 77 bp and the band below 100 bp corresponds to it. The expected length of rs73064425 was 532 bp and a band below 600 bp corresponds to it.

FIG. 4(c) shows amplification of SNPs employing the following comparative primers:

TABLE E

| SNP | Name | Sequence (5′-3′) |
|---|---|---|
| rs 10735079 | fwd_rs10735079_ | ACATAAAATACAAGAT |
| | rc | TTTGCAGAATTTTG |
| | | [SEQ ID No. 19] |
| | rvs_rs_10735079_ | GTTTCCCTCAGCCACT |
| | rc | GCC |
| | | [SEQ ID No. 20] |
| rs2109069 | fwd_rs2109069_ | GGGTTTTGGGCTCCAA |
| | rc | GAGATTTGGG |
| | | [SEQ ID No. 21] |
| | rvs_rs2109069 | CCAGCCTGGGTAAC |
| | | AGAGCAAGAC |
| | | [SEQ ID No. 22] |
| rs73064425 | fwd_rs73064425 | GAAGTAGTTTTCTCA |
| | | TTTTTAAATGACAA |
| | | AATTTA |
| | | [SEQ ID No. 23] |
| | rvs_rs73064425 | GCCAGCCTGGGCAAC |
| | | AGAG |
| | | [SEQ ID No. 24] |

Results of the amplification employing comparative primers of Table D are provided in FIG. 4(c). Lane-1 shows PCR product amplified using the comparative primer set in Table D designed for the amplification of regions of the genomic DNA that harbors SNP rs10735079. The primer sets were designed such that the length of the amplified product was 140 bp. There was no detectable band the gel; hence the primer set was determined to be inefficient. Lane-3 shows PCR product amplified using the comparative primer set in Table D designed for the amplification of regions of the genomic DNA that harbors SNP rs2109069. The primer sets were designed such that the length of the amplified product was 162 bp. There is no detectable band in the gel; hence the primer set was determined to be inefficient. Lane-5 has PCR product amplified using the comparative primer set in Table D for the amplification of regions of the genomic DNA that harbors SNP rs73064425. There was no detectable band in the gel; hence the primer set was determined to be inefficient. Lane 7 shows the 100 bp ladder marker.

Based on the above, the primers listed in Table C were shortlisted for facilitating amplification of genomic DNA.

### Example 4: SNP Detection in Genomic DNA

The ability of the selected guides to recognize the SNPs of interest [rs10735079, rs2109069, and rs73064425] in genomic DNA was analyzed on DNA purchased from Coriell Institute for Medical research. These genomic DNA belong to the international HapMap project. The genotype of this sample at the selected SNP loci from the sequencing data is available in publicly available databases. The ability of the assay incorporating the selected guides to accurately predict the genotype of each SNP was studied.

Initially, the genomic DNA was subjected to amplification using the primers as set out in Table C. Briefly, amplification was done using 50 ng of genomic DNA in 50 uL reaction volume using 250 nM of each primer. The amplification was done using OneTaq® 2× Master Mix with Standard Buffer (M0482S, NEB USA). The amplification conditions used were following: Initial heat denaturation at about 94° C. for about 5 minutes; 30 cycles of beat denaturation at about 94° C. for about 30 seconds, annealing at about 60° C. for about 30 seconds, extension at about 68° C. for about 30 seconds; and final extension for about 5 minutes at about 68° C. For amplification of SNP in individual tubes forward and reverse primers for the SNP were added to the tube. For multiplexed amplification of all SNPs in a single tube, forward and reverse primer for all SNPs was added to the single tube.

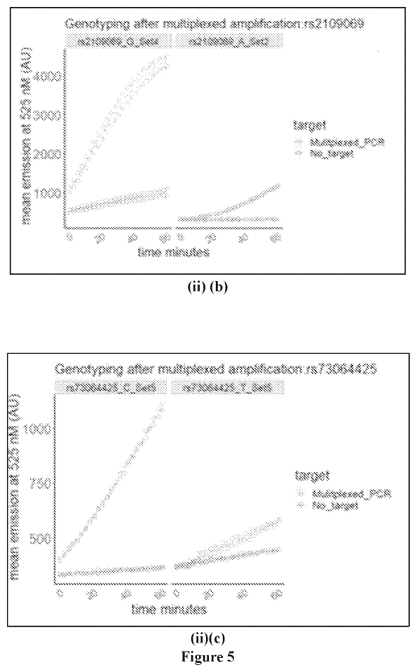

Next, the ability of the selected guides to genotype genomic DNA (subjected to individual amplification (5(i) and multiplexed amplification 5(ii)) was studied. Specifically, the ability of the assay incorporating the selected guides to accurately predict the genotype of each SNP was analyzed. FIGS. 5(i and ii) shows the trans-cleavage assays done to ascertain the genotype of NA12878 using the guide pair designed for each SNP. First the region harboring the SNP of interest was subjected to amplification (5(i)) or multiplexed amplification (5(ii)) using the selected primer set and the trans-cleavage assay was done in the presence of this PCR product as target with each guide of the guide pair corresponding to the SNPs. A quantitative criterion was set up to assign the genotype using the trans-cleavage rates of the selected guide pair as described ahead. “selectivity ratio” was calculated for each guide for the guide pair of a SNP. The formula for calculating the selectivity ratio is shown below.

Selectivity ratio alternate guide=(Intensity in the presence of the alternative allele specific guide at 30 minutes from the start of data acquisition−Intensity in the presence of the alternative allele specific guide at 10 minutes from the start of data acquisition)/Intensity in the presence of the risk allele specific guide at 30 minutes from the start of data acquisition−Intensity in the presence of the risk allele specific guide 10 minutes from the start of data acquisition).

Selectivity ratio risk guide=(Intensity in the presence of the risk allele specific guide at 30 minutes from the start of data acquisition−Intensity in the presence of the risk allele specific guide at 10 minutes from the start of data acquisition)/Intensity in the presence of the alternative allele specific guide at 30 minutes from the start of data acquisition−Intensity in the presence of the alternative allele specific guide 10 minutes from the start of data acquisition).

The selectivity ratio for the guide that gives higher signal was calculated, and the genotyping was done as described below.

Selectivity ratio>2 was determined to be indicative homozygous for that allele or variant.

Selectivity ratio>0.5-<2 was determined to be indicative of heterozygous for that allele or variant.

Selectivity ratio<0.5 was determined to be indicative of homozygous for the other allele.

The results of trans-cleavage experiments on samples subjected to amplification and multiplexed amplification showed similar results. Elaboration on said results, below, is provided for one of the two—FIG. 5(ii) depicting results of the trans-cleavage assay on the samples subjected to multiplexed amplification, for reasons of brevity.

FIG. 5(ii)(a) shows the trans-cleavage data for the guide pair selected for SNP rs10735079 in the presence of the PCR product obtained using the primer set designed to amplify the region harboring SNP rs10735079. The left panel shows the trans-cleavage rates of the CRISPR-Cas complex containing variant G selective guide (alternative allele), and the right panel shows the trans-cleavage rates of the CRISPR-Cas complex containing variant A selective guide (risk allele) in the presence of the PCR product of NA12878. As expected in the absence of PCR products, none of the guides showed trans-cleavage. The rate of trans cleavage is higher in the presence of G selective guide than A selective guide. The selectivity ratio for variant G selective guide was calculated and it was 13 and hence the genotype at this location was determined to be GG or homozygous for the alternative allele

FIG. 5(ii)(b) shows the trans-cleavage data for the guide pair selected for SNP rs2109069 in the presence of the PCR product obtained using the primer set designed to amplify the region harboring SNP rs2109069. The left panel shows the trans-cleavage rates of the LbaCas12a complex containing variant G selective (alternative), and the right panel shows the trans-cleavage rates of the LbaCas12a complex containing variant A (risk allele) selective guide in the presence of the PCR product of NA12878. As expected in the absence of PCR products, none of the guides showed trans-cleavage The variant G selective guide showed faster rate of trans cleavage than variant A selective guide. The selectivity ratio for variant G selective guide was calculated and it was 19. Hence the genotype at this location is GG or homozygous for the alternative allele.

FIG. 5(ii)(c) shows the trans-cleavage data for the guide pair selected for SNP rs73064425 in the presence of the PCR product obtained using the primer set designed to amplify the region harboring SNP rs73064425. The left panel shows the trans-cleavage rates of the CRISPR-Cas complex containing variant C selective (alternative allele), and the right panel shows the trans-cleavage rates of the CRISPR-Cas complex containing variant T (risk allele) selective guide in the presence of the PCR product of NA12878. As expected in the absence of PCR products, none of the guides showed trans-cleavage. The C selective guide showed faster trans-cleavage than T selective guide. The selectivity ratio for C-selective was calculated and it was 3.13, and hence the genotype at this location was determined to be CC or homozygous for the alternative allele.

The above therefore shows that the primer and guide pairs of the present disclosure are able to facilitate detection of the risk and/or alternate alleles for the SNPs of interest in genomic DNA samples with high selectivity

The foregoing description fully reveals the general nature of the embodiments herein that others can, by applying current knowledge, readily modify and/or adapt for various applications such specific embodiments without departing from the general concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments in this disclosure have been described in terms of preferred embodiments, those skilled in the art will recognize that the embodiments herein can be practiced with modification within the spirit and scope of the embodiments as described herein, without departing from the principles of the disclosure.

Any discussion of documents, acts, materials, devices, articles and the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

1. An assay device for determining genetic predisposition to severe forms of SARS-CoV2 infection, the assay device comprising at least one CRISPR-Cas system for genotyping at least one Single Nucleotide Polymorphism (SNP) selected from a group comprising rs10735079, rs2109069 and rs73064425 or any combination thereof in a biological sample.

2. The assay device as claimed in claim 1, wherein the device further comprises separate CRISPR-Cas systems for detection of each SNP.

3. The assay device as claimed in claim 1, wherein the CRISPR-Cas system comprises sgRNA sequence selected from a group comprising sequences represented by SEQ ID Nos. 1-6.

4. The assay device as claimed in claim 1, wherein the CRISPR-Cas system has sgRNA sequence selected from a group comprising sequences represented by SEQ ID Nos. 1-3 for confirming presence of at least one risk allele for at least one SNP selected from a group comprising rs10735079, rs2109069 and rs73064425 or any combination thereof.

5. The assay device as claimed in claim 1, wherein the CRISPR-Cas system comprises sgRNA sequence selected from a group comprising sequences represented by SEQ ID Nos. 4-6 for confirming presence of at least one alternate allele for at least one SNP selected from a group comprising rs10735079, rs2109069 and rs73064425 or any combination thereof.

6. The assay device as claimed in claim 1, wherein the CRISPR-Cas system comprises Cas nuclease selected from a group comprising dCas9, Cas9, Cas-Phi, Cas12, Cas12a, Cas12b and Cas13a or any combination thereof.

7. The assay device as claimed in claim 6, wherein the Cas nuclease is Cas12a, and wherein the detection is facilitated by trans-cleavage mechanism of the Cas12a nuclease.

8. The assay device as claimed in claim 1, further comprising at least one reporter molecule in a format of F—(N)ₙ-Q or Q-(N)ₙ—F, wherein;
F is a fluorescent reporter molecule,
N is selected from A, G, T, C, rA, rG, rT and rC, and
Q is a quencher.

9. The assay device as claimed in claim 8, wherein:
the fluorescent reporter molecule, F, is selected from a group comprising SYT09, fluorescein, rhodamine, rhodoamine600, R-phycoerythrin, and Texas Red or any combination thereof,
the quencher, Q, is selected from a group comprising black hole quencher (BHQ), Iowa black and 4-(dimethylaminoazo)benzene-4-carboxylic acid (DABCYL), and
n ranges from 6 to 15.

10. The assay device as claimed in claim 1, wherein the at least one CRISPR-Cas system is immobilized on a surface of the assay device.

11. A method of detecting genetic predisposition to severe forms of SARS-CoV2, the method comprising adding a biological sample to the assay device as claimed in claim 1 to obtain a detectable signal.

12. The method as claimed in claim 11, wherein the sample comprises nucleic acid, and wherein the nucleic acid is subjected to amplification before addition to the assay device.

13. The method as claimed in claim 12, wherein the amplification is performed by Polymerase Chain Reaction (PCR), and wherein at least one primer for the PCR amplification is selected from a group comprising sequences represented by SEQ ID Nos. 7-12.

14. The assay device as claimed in claim 1, wherein genetic predisposition to severe forms of SARS-CoV2 infection is determined by presence of risk and/or at least one alternate allele for SNPs selected from a group comprising rs10735079, rs2109069 and rs73064425 or any combination thereof in the biological sample.

15. An sgRNA sequence against loci of at least one SNP selected from a group comprising rs10735079, rs2109069 and rs73064425 or any combination thereof.

16. The sgRNA sequence as claimed in claim 15, comprising a sequence selected from a group comprising sequences represented by SEQ ID Nos. 1-6.

17. The sgRNA sequence as claimed in claim 15, for use in detecting genetic predisposition to severe forms of SARS-CoV2.

18. A device for determining genetic predisposition to severe forms of SARS-CoV2 infection, the device comprising at least one CRISPR-Cas system for detection of at least one Single Nucleotide Polymorphism (SNP) selected from a group comprising rs10735079, rs2109069 and rs73064425 or any combination thereof in a biological sample.

19. A kit comprising the assay device as claimed in claim 1, and an instruction manual.

20. The kit as claimed in claim 19, further comprising at least one component selected from a group comprising a multi-well plate, at least one reaction buffer, at least one unit for sample procurement, at least one unit for application of the sample to the assay device, at least one primer, dNTPs, at least one polymerase enzyme and at least one reporter system or any combination thereof.

21. The method as claimed in claim 11, wherein genetic predisposition to severe forms of SARS-CoV2 infection is determined by presence of risk and/or at least one alternate allele for SNPs selected from a group comprising rs10735079, rs2109069 and rs73064425 or any combination thereof in the biological sample.

22. A device for determining genetic predisposition to severe forms of SARS-CoV2 infection, the device comprising the sgRNA sequence as claimed in claim 15.

23. A device for determining genetic predisposition to severe forms of SARS-CoV2 infection, the device comprising a CRISPR-Cas complex comprising the sgRNA sequence as claimed in claim 15 and Cas12a nuclease.

24. A kit comprising the sgRNA sequence as claimed in claim 15, and an instruction manual.

25. The kit as claimed in claim 24, further comprising at least one component selected from a group comprising a multi-well plate, at least one reaction buffer, at least one unit for sample procurement, at least one unit for application of the sample to an assay device, at least one primer, dNTPs, at least one polymerase enzyme and at least one reporter system or any combination thereof.
